(19) United States
(12) Patent Application Publication
(10) Pub. No.: US 2023/0301306 A1
(43) Pub. Date: Sep. 28, 2023

(54) AN AEROSOL COMPOSITION FOR ELIMINATING PATHOGENIC MICROORGANISMS

(71) Applicant: LIFECLEAN INTERNATIONAL AB, Uddevalla (SE)

(72) Inventors: Bo LENNERNÄS, Uddevalla (SE); Carl-Gustav MILLINGER, Stockholm (SE)

(21) Appl. No.: 18/041,419

(22) PCT Filed: Aug. 17, 2021
(86) PCT No.: PCT/SE2021/050800

§ 371 Date: Feb. 13, 2023

(30) Foreign Application Priority Data
Aug. 17, 2020 (SE) 2050958-4

(51) Int. Cl. A01N 59/00; A61L 9/14; A01N 25/06; A01P 1/00

(52) U.S. Cl. CPC A01N 59/00; A61L 9/14; A01N 25/06; A01P 1/00; A61L 2101/06

(57) ABSTRACT

Disclosed herein is a method of eliminating pathogens with an aerosolized composition with polar or charged droplets comprising chlorine dioxide The aerosolized composition is generated from an acidic chlorine dioxide composition comprising at least one charged surfactant.

### TECHNICAL FIELD

The present invention relates generally to disinfection of target regions aerosolized composition comprising chlorine dioxide with an amplified anti-microbial effect.

### BACKGROUND OF THE INVENTION

It is a well-known fact that viable pathogenic viruses exist in the micrometer sized droplets exhaled or ejected from the respiratory system of infected subjects and that the droplets may circulate in the air, especially in confined spaces, for a sufficient time to spread the infection by inhalation. To solve such problems, JP 2002-272827 describes uses of chlorine dioxide gas for preventing from infections from floating virus, for example virus arriving from air conditioning systems.

Chlorine dioxide is an established disinfectant and has been used extensively for water cleaning, water processing and industrial sanitization. It is known to be a labile, volatile compound and to be a powerful oxidizing agent which in many aspects limits its applicability. It is also highly irritating in high concentrations and authorities like The Occupational Safety and Health Administration (OSHA) in the US has set an 8-hour permissible exposure limit to 0.1 ppm (0.28 mg per cubic meter) in air.

WO 03/063917 describes disinfection of surfaces with an electrostatically charged aerosol of a disinfection solution comprising chlorine dioxide and efficacy is suggested for elimination of spore forming microorganisms such as Bacillus anthracis. However, no particular or fully outlined composition of disinfection solution is disclosed and no results of the disinfection is demonstrated.

WO 2014/129956 describes acidic disinfection compositions comprising 100 to 2000 ppm chlorine dioxide with improved efficacy to eliminate spore forming bacteria on surfaces supported by a complementary system of surfactants.

EP 1955719 B1 describes uses of chlorine dioxide gas at a concentration 0.0001 to 0.1 ppm to deactivate respiratory virus in spaces and to treat infected living organisms infected by a respiratory virus. For these purposes, a low level chlorine dioxide gas inhalation exposure chamber was outlined by A Akamatsu et al. in Journal of Occupational Medicine and Toxicology, 2012, 7:2, page 1-8, in order to expose test animals and confirm the potential safety of sub 0.1 ppm level exposures. The antiviral activity of chlorine dioxide is further explained and documented in for example, K Kaly-Kullai et al Physiology International 2020, 107 1, 1-11; and S Andreu et al. Viruses, 2021, 13(3). 531ff.

It remains a need for improved chlorine dioxide based compositions and delivery forms to safely and effectively eliminate pathogenic microorganisms including virus from air also in populated spaces that also potentially are useful in therapy in order to treat subjects suffering from various grades of virus infection in the respiratory system.

A conventional method of disinfecting smaller rooms at hospitals, care centers and emergency vehicles is to use gaseous or diffused peroxides such as hydrogen peroxide. However, these methods have some general drawbacks from the fact that unpleasant odors may be generated and that sensitive electronic equipment may become compromised. It is therefore a need to develop alternative effective disinfection methods based on chlorine dioxide.

### DESCRIPTION OF THE INVENTION

It is a general object of the invention to provide methods of disinfection of rooms and other spaces that eliminates a wide range of pathogens with chlorine dioxide.

It is an object of the invention provide an aerosol composition comprising chlorine dioxide in amounts for effective disinfection of selected rooms or spaces from a wide range of pathogens or comprising amounts of chlorine dioxide suitable and effective in therapies.

It is also an object of the invention to provide an aerosol composition with a polarity or charge to support its distribution in the selected space

In a general aspect, the present invention is directed to a method of eliminating pathogenic microorganisms that comprises (a) to provide an aerosolizable aqueous composition with a physiologically acceptable pH below 7.5, comprising an effective amount of chlorine dioxide (CD), at least one charged surfactant operable at an acidic pH and in the presence of CD, and optionally one or more suitable excipients; (b) to form a polar or charged aerosol from said composition with a droplet size of 1 to 120 μm; and (c) to allow the aerosol composition to distribute in a region targeted for pathogen elimination.

Pathogenic microorganisms in this general aspect typically are bacteria such as Mycobacterium tuberculosis, Bordetella pertussis, fungi such as aspergillus, virus, respiratory virus, such as types of influenza virus and coronavirus, rhinovirus and calicivirus. In one embodiment of the method the pathogenic microorganism is a respiratory virus, present both in circulating aerosol droplets and distributed on surfaces from infected subjects in rooms with limited air circulation.

The term operable surfactant has the meaning that the surfactant has surfactant capacity, such as wetting and/or solubilization at the given pH, in the presence of CD and in the charged aerosol droplets, so it can contribute to or assist with pathogen elimination.

The term target region for pathogen elimination or disinfection has in the context of the present invention the meaning of any closed space, room or compartment, wherein viable pathogens, for example, circulate in exhaled or excreted aerosol-like droplets and/or are present on surfaces.

The term distribute in a region targeted for pathogen elimination has the meaning that the described aerosolized composition actively or passively is distributed to the region, which may include conventional electrostatic spray arrangements, nebulizers or other means.

Preferably, according to the method the droplets are positively charged, preferably to a level of at least 1 millicoulomb per ml aerosolizable composition, for example 1 to 10 millicoulomb per ml aerosolizable composition.

In one aspect, the method is adapted to be performed in populated target regions and thereby admits 0.1 ppm CD (0.3 mg/m3), or less, in order to comply with general hygienic prescriptions for CD.

In other aspects, the method can be performed with substantially higher levels of CD in order the accomplish an effective disinfection in short time periods by varying exposure times. Within the defined CD concentration range of 10 to 2000 ppm of the aerosolizable composition, several levels of efficacy can be reached as necessary to obtain a desired disinfection result.

In one aspect, the method is performed as a disinfection procedure of a compartment or room and an aerosol is introduced with 20 to 60 μm average droplet size, preferably about 40 μm generated from an aqueous aerosolizable composition comprising 200 to 600 ppm chlorine dioxide with a pH of 1 to 3, preferably 1.8 and comprising one or more, preferably at least two, charged surfactants, as described herein.

In another general aspect, the invention is directed to an aerosol composition of droplets having size of 1 to 120 μm for eliminating pathogenic microorganisms, wherein the droplets are polar and/or charged and said composition is aerosolized from an acidic aqueous composition with a pH of less 7, comprising 10 to 2000 ppm chlorine dioxide (CD); at least one charged surfactant; an acid and optionally other excipients.

Preferably, according to this general aspect, the at least one surfactant is a hydrocarbon ionic surfactant selected from cationic surfactants and anionic surfactants.

The hydrocarbon ionic surfactant can be selected from at least one of amine oxides, alkyl sulphates, alkyl sulphonates, alkyl aryl sulphonates, aryl sulphonates and salts thereof. When the surfactant is at least one amine oxide, preferably the alkyl groups have 6 to 18 carbon atoms,

In one aspect, the surfactant includes two or more amine oxides with alkyl groups having 6 to 18 carbon atoms, wherein at least two amine oxides have alkyl groups with a difference in hydrocarbon chain length of at least four carbon atoms.

When the surfactant is at least one amine oxide, the surfactant is preferably present in amount of less than 2% (v/v), preferably in an amount of 0.1 to 2% (v/v).

In a particular embodiment of the aerosolizable acidic aqueous composition, the pH is from 1 to 5, the amount of CD is from 100 to 500 ppm and the surfactant is a combination of amine oxides with different length in carbon chains, preferably one amine oxide with 8 carbon atoms and an amine oxide with 12 carbon atoms, preferably the amine oxides are octyl dimethylamine oxide and lauryl dimethylamine oxide. In particularly preferred embodiment, the pH is 1.8-2.0, the amount of CD is nominally 300 ppm and the surfactant is present in an amount of 0.1 to 0.2% (v/v).

Preferably, the aerosol droplets as described are electrostatically charged, more preferably electrostatically positively charged.

Preferably, the droplets have a charge of at least 1 millicoulomb per ml aqueous composition and more suitably 1 to 10 millicoulomb per ml aqueous composition.

The method and aerosol compositions according to the invention are exemplified in disinfection applications, but are considered useful in the therapeutic applications outlined in EP 1955719 to treat infections and complications arriving from severe infections respiratory pathogens, as exemplified by ARDS (Adult Respiratory Distress Syndrome), when administered in therapeutically effective acceptable regimens. Accordingly, the present invention shall be regarded to claim coverage of both disinfecting and therapeutic applications.

### DETAILED AND EXEMPLIFYING DESCRIPTION OF THE INVENTION

### Example 1

LifeClean® (from LifeClean International AB, Uddevalla Sweden) is a commercial chlorine dioxide product with documented surface disinfection effect for a wide range of bacteria, virus and fungi, see https://www.lifeclean.se/media/203776/lifeclean-microbiological-efficacy-summary-20200416.pdf.

LifeClean® (LC) comprises approximately 300 ppm of chlorine dioxide (CD) in an acidic composition with a pH of about 1.8 and includes a system of the cationic surfactants octyl dimethylamine oxide and lauryl dimethylamine oxide. The surfactant system of LC has been selected so that it remains ionic at the selected, effective pH, is resistant to chlorine dioxide and exhibits complementary wetting and solubilizing effects.

The present experiments were performed to assess the efficacy of LC to eliminate pathogens in closed potentially contaminated environments in comparison with conventional chlorine dioxide formulations including gaseous chlorine dioxide and thereby assess advantageous embodiments of the invention.

For this purpose, a bacterial application was performed in the confined space of a 97-liter aquarium. Luminescent bacteria (Aliivibrio fischeri) were obtained with the product LumoStix® and were mixed with buffer according to instructions and placed on absorbent pads for location at predetermined positions in the aquarium. For the following experiments luminescence was measured with a luminometer type Kikkoman PD30. This equipment was provided with a nebulizing spray device type Victory Ecostatic capable of generating both electrostatically positively charged and uncharged aerosols with a particle size of 40 to 110 microns.

For the experiments, the following agents were tested:
LC is 18 ml LifeClean product distributed on a plate in the aquarium
LCa is LifeClean product applied as non-electrostatic spray/aerosol
LCa+ is LifeClean product applied as electrostatic spray/aerosol
Xa is a pH-neutral pure water solution of approx.300 ppm chlorine dioxide applied as non-electrostatic spray/aerosol
Xa+ is a pH-neutral pure water solution of approx.300 ppm chlorine dioxide applied as electrostatic spray/aerosol.

In all experiments the luminescence of the bacteria Aliivibrio fischeri was compared to the luminescence immediately before the chlorine dioxide exposure in a relative measurement, using the luminometer. In the first experiments (Study 1-2), the stability of the LumoStix® arrangement including the buffer was studied and the effect was compared to LC alone. The buffer and bacteria were mixed according to the LumoStix® instructions of use and a declination of luminescence was noted. In these tests, the bacteria were exposed to the disinfectant agent (ClO2) after bacteria application on the LumoStix® absorbent pads. In study 1, 18 ml LC was placed in the aquarium on a plate. This setting has resemblance to applying ClO2 gas only to the test object. No aerosol and/or no additional product formulation additives. This corresponds to 10 seconds blow using the spray device producing LCa. The bacteria LumoStix® absorbent pads were exposed to the closed aquarium for 30 seconds. The aim of these studies was to check any possible advantage for further evaluation. In Study 4, LCa was compared to LCa+. The effect on the bacteria was drastic so blow time was reduced to five seconds and incubation of 15 seconds. Study 5 was a copy of Study 4, but the agent is Xa/Xa+ instead of LC/LCa/LCa+

The following experiments were performed:

Study 1 was an evaluation of if the LumoStix® arrangement was suitable for a LC test and a comparison of LCa versus LC. Study 2 was performed to check stability of the bacteria buffer. Study 3 was a comparison of the performance of LCa versus LC. Study 4 was a comparison of the performance of LCa+ versus LCa. Study 5 was a comparison of the performance Xa versus Xa+.

The results from Study 1 are presented in Table 1, below, and shows a better effect of 18 ml LCa compared to LC.

Table 2, below, presents the results form Study 4 and demonstrates the effect on bacterial survival from the exposure of LCa in three different exposures.

Table 3 below presents the results form Study 4 and demonstrates the effect on bacterial survival from the exposure of LCa+ in three different exposures. It is evident comparing Table 2 and 3, that LCa+ is more efficient in eliminating bacterial in the present test model than LCa.

Tables 4 and 5, below presents the results from Study 5 and demonstrate the effect of Xa and Xa+, respectively. In comparison with the results of Tables 1 and 3, it is evident that LCa and LCa+ is more efficacious than Xa/Xa+ in in the present test model.

In conclusion, from Studies (1-2) it was found that the buffer needs to be room temperate (approximately 1 hour) and measurements should be performed approximately 3 minutes after any exposure of chlorine dioxide. It was also shown that LCa has a more pronounced effect than LC, see Table 1. The bacteria were suitably sensitive for chlorine dioxide and LumoStix® is accordingly a suitable system for the following studies 3-5.

From the Tables 2-5, showing Studies 3-5, it can be concluded that LCa is more effective compared to LC during the time period in the experiment (Study 3). In Study 4, LCa+ was 1,7-1,8 more effective than LCa. This effect could not be noted in Study 5, comparing Xa versus Xa+ without the additives of LifeClean®.

It is shown that the survival fraction, measured as a reduction of bacteria light emission, was higher using LCa compered to LC. The effect was enhanced by electrostatic charging of LC, i.e., generating LCa+. The effect on electrostatic charging could not be shown using pure ClO2 (Xa+). It was also noted that the luminescence further declined in the tests with LCa and LCa+, but not LC and Xa/Xa+

In summary the methods and the aerosol compositions according to the present invention, based on a chlorine dioxide composition comprising at least one charged surface-active component, have an improved efficacy in eliminating microorganisms.

TABLE 1

| Study 1 | LCa | LC |
|---|---|---|
| Survival fraction | 48% | 72% |
| | 18% | 67% |
| | 62% | 85% |
| Mean | 43% | 75% |

TABLE 2

| LCa | 1 | 2 | 3 |
|---|---|---|---|
| Start | 1200 | 223 | 274 |
| Min 1 | 1078 | — | |
| Min 2 | 1138 | 241 | |
| Min 3 | 1105 | 252 | |
| Mean | 1130 | 239 | |
| After exposure | 108 | 84 | 95 |
| +15 sek | 62 | | 84 |
| +15 sek | 38 | | 81 |
| Survival fraction | 9.6% | 35% | 34% |
| Mean | | | 26% |

TABLE 3

| LCa+ | | | |
|---|---|---|---|
| Start | 968 | 227 | 264 |
| Min 1 | 717 | 239 | |
| Min 2 | 639 | — | 241 |
| Min 3 | 651 | 235 | 240 |
| Mean | 743 | 233 | 248 |
| After exposure | 39 | 42 | 51 |
| +15 sek | 16 | 26 | 36 |
| +15 sek | 11 | | 39 |
| Survival fraction | | | 20% |
| Mean | 5.2% | 18% | 14.4% |

TABLE 4

| Xa | | | |
|---|---|---|---|
| Start | 484 | 232 | 210 |
| Min 1 | 453 | 236 | 201 |
| Min 2 | 417 | 232 | 193 |
| Min 3 | 440 | 164 | 192 |
| Mean | 448 | 216 | 199 |
| | | 233 | |
| After exposure | 148 | 109 | 75 |
| +15 sek | 150 | 111 | — |
| +15 sek | 148 | 110 | 74 |
| Survival fraction | 33% | 48 | 37% |
| Mean | | | 39% |

TABLE 5

| Xa+ | | | |
|---|---|---|---|
| Start | 288 | 200 | 344 |
| Min 1 | 277 | 233 | 321 |
| Min 2 | 265 | — | 308 |
| Min 3 | 259 | 218 | 302 |
| Mean | 272 | 217 | 318 |
| After exposure | 91 | 113 | 97 |
| +15 sek | 91 | 106 | — |
| +15 sek | 90 | 108 | 93 |
| Survival fraction | 33% | 52% | 30% |
| Mean | | | 38% |

### Example 2

This example demonstrates the efficacy of the methods and composition of the invention to eliminate pathogens in a targeted space represented by the interior of an emergency vehicle.

E Coli K12 (1.5 cm culture, Heraco) were suspended in NaCl and grafted to agar plates comprising horse blood with a conventional needle in of 10 μm in 3 rounds and conventionally distributed in zig-zag patterns, while rotating the agar plate ⅓ . The so prepared agar plates acclimatized for an hour and incubated 24 hours. 4 plates were kept as control and 7 were placed in different parts in the compartment of the emergency vehicle.

LifeClean® (LC) comprising approximately 300 ppm of chlorine dioxide was introduced in the 8 m³ volume of the emergency vehicle compartment with the nebulizer Victory Ecostatic of Example 1 with a 40 micron nozzle providing 3.4 oz/min or 97 ml LC per minute. The nebulizer was operated for 1 minute 6 times between breaks of 30 seconds, followed by 3 minutes exposure giving a total exposure time of 11-12 minutes. The estimated theoretically maximum concentration of gaseous chlorine dioxide in the compartment was about 20 ppm. In practice, chlorine dioxide will present both as gas and dissolved in the aerosol droplets during the exposure.

After exposure were both control plates and exposed plates aerated before incubation and CFU (colony forming unit) count to avoid chlorine dioxide to affect control.

The results after CFU count are demonstrated in Table 6. All control plates had a CFU of>400.

TABLE 6

| Location in vehicle of plate | CFU Count |
|---|---|
| 1. Front seat right | 15 |
| 2. Shelf above driver | 23 |
| 3. Rear stretcher | 40 |
| 4. Right upper cabinet | 6 |
| 5. Left upper cabinet | 28 |
| 6. Right shelf medium height | 2 |
| 7. Front stretcher | 100+ |

Even if agar plate 7 is an outlier, the results of Table 6 show that the method and composition according to the invention has clear efficacy in sanitary procedure in eliminating pathogens in potentially infected emergency vehicle. The results indicate that 92 to 95% elimination is reached. Accordingly, the invention will be useful in effectively eliminating a wide range of pathogens in a surprisingly fast an expedient way and will be useful in many clinical and public settings where airborne pathogens are expected to contaminate closed spaces and rooms.

1. An aerosol composition of droplets having size of 1 to 120 μm for eliminating pathogenic microorganisms, wherein the droplets are polar and/or charged and said composition is aerosolized from an aqueous composition with a pH of less than 7.5, comprising 10 to 2000 ppm chlorine dioxide (CD); at least one charged surfactant; an acid and optionally other excipients.

2. The composition according to claim 1, wherein the at least one surfactant is a hydrocarbon ionic surfactant selected from cationic surfactants and anionic surfactants.

3. The composition according to claim 2, wherein the hydrocarbon ionic surfactant is selected from at least one of amine oxides, alkyl sulphates, alkyl sulphonates, alkyl aryl sulphonates, aryl sulphonates and salts thereof.

4. The composition according to claim 3, wherein the surfactant is at least one amine oxide, preferably with alkyl groups having 6 to 18 carbon atoms, more preferably the surfactant includes two or more amine oxides with alkyl groups having 6 to 18 carbon atoms, wherein at least two amine oxides have alkyl groups with a difference in hydrocarbon chain length of at least four carbon atoms.

5. The composition according to claim 3, wherein the surfactant is present in amount of less than 2% (v/v), preferably in an amount of 0.1 to 2% (v/v).

6. The composition according to claim 4, wherein the pH is from 1 to 5, the amount of CD is from 100 to 500 ppm and the surfactant is a combination of an amine oxide with 8 carbon atoms and an amine oxide with 12 carbon atoms, preferably the amine oxides are octyl dimethylamine oxide and lauryl dimethylamine oxide.

7. The composition according to claim 1, wherein the pH is from 1.8 to 2.0, the amount of CD is 300 ppm and the surfactant is present in an amount of 0.1 to 0.2% (v/v).

8. The composition according to claim 1, wherein the droplets are electrostatically charged.

9. The composition according to claim 1, wherein the droplets are positively charged.

10. The composition according to claim 1, wherein the droplets have a charge of at least 1 millicoulomb per ml aqueous composition, preferably 1 to 10 millicoulomb per ml aqueous composition.

11. A method of eliminating pathogenic microorganisms comprising:
(i) providing an aerosolizable aqueous composition having a pH below 7.5, comprising an effective amount of chlorine dioxide (CD), at least one charged surfactant operable at an acidic pH and in the presence of CD, and optionally one or more suitable excipients;
(ii) forming a polar and/or charged aerosol with a droplet size of 1 to 120 μm; and
(iii) allowing the aerosol to distribute in a region targeted for pathogen elimination.

12. The method according to claim 11, comprising electrostatically charging the aerosol.

13. The method according to claim 12, wherein the droplets obtain a positive charge, preferably of 1 to 10 millicoulomb per ml aerosolizable composition.

14. The method according to claim 11, wherein the pathogenic microorganism is an airborne pathogen, preferably a respiratory virus.

15. The method according to claim 11, admitting 0.1 ppm CD, or less, in a populated target region.

16. The method according to claim 11, wherein the aerosol comprises 10 to 2000 ppm chlorine dioxide (CD). composition in accordance with any one of claims 1 to 10.
